(19) United States
(12) Patent Application Publication
(10) Pub. No.: US 2025/0281068 A1
(43) Pub. Date: Sep. 11, 2025

(54) Modular Respiratory Sensor Integration Block System

(71) Applicant: Texas Tech University System, Lubbock, TX (US)

(72) Inventors: Robert V. Duncan, Lubbock, TX (US); Annette L. Sobel, Lubbock, TX (US); Shelby Lacouture, Levelland, TX (US); Paul D. Walter, Lubbock, TX (US)

(21) Appl. No.: 18/860,033

(22) PCT Filed: Apr. 17, 2023
(86) PCT No.: PCT/US2023/065831

§ 371 Date: Oct. 25, 2024

Related U.S. Application Data
(60) Provisional application No. 63/334,254, filed on Apr. 25, 2022.

(51) Int. Cl. A61B 5/091; A61B 5/00; A61B 5/08; A61B 5/097; A61B 5/1455

(52) U.S. Cl. CPC A61B 5/091; A61B 5/082; A61B 5/097; A61B 5/14552; A61B 5/4848; A61B 5/6803

(57) ABSTRACT

The present invention includes sensor integrated block (SIB) system and method of using the same for real-time respiratory data comprising: a chamber comprising an inside, an inlet and outlet for air, wherein two or more first sensors are in fluid communication with the inside of the chamber, wherein the two or more sensors are selected from an oxygen sensor (2), a carbon dioxide sensor (3), a pressure sensor (4), and a temperature sensor (5); one or more second sensors selected from an SpO₂ sensor (6) or a PaCO₂ sensor; and a processor connected to each of the first and second sensors, and wherein the SIB system measures in real-time respiratory pressure and the inhalation and exhalation volumes of each breath of a patient.

### CROSS-REFERENCE TO RELATED APPLICATIONS

This application claims priority to U.S. Provisional Application Ser. No. 63/334,254, filed Apr. 25, 2022, the entire contents of which are incorporated herein by reference.

### TECHNICAL FIELD OF THE INVENTION

The present invention relates in general to the field of respirators or breathing devices, and more particularly, to a modular respiratory sensor integration block system and its use as a medical diagnostic device.

### STATEMENT OF FEDERALLY FUNDED RESEARCH

None.

### BACKGROUND OF THE INVENTION

Without limiting the scope of the invention, its background is described in connection with respirators or breathing devices.

Respiratory assist devices help patients in need of support for breathing, removal of carbon dioxide, and therapy to reduce atrophy of abdominal wall muscles. Demand for these devices has risen significantly during the pandemic as they are invaluable for treating patients severely impacted by the COVID-19 pandemic. Mechanical ventilation is required when it becomes very difficult for a patient to breathe or get enough oxygen into their blood, which indicates a patient might be experiencing respiratory failure. Mechanical ventilators are medical devices that move air in and out of the lungs to keep the patient alive. Some ventilators provide support to patients who do not require complex critical care ventilators and these typically consist of a flexible breathing circuit, a control system, monitors, and alarms.

Other systems might also consist of oxygen accumulators, heated humidifiers, or heat and moisture exchangers to improve patient comfort. Long-term and emergency care devices use positive pressure to deliver gas to the lungs at normal breathing rates and tidal volumes through an endotracheal tube, a tracheostomy cannula, or a mask. Another assistive respiratory device is the Continuous positive airway pressure therapy (CPAP), which uses a machine to treat patients suffering from obstructive sleep apnea (OSA). A CPAP machine increases air pressure in the throat so that the airway doesn't collapse during inhalation.

Although respiratory support devices have advanced significantly over the last few decades, there are still aspects of the systems that need improvement. Respiratory monitoring is important and could be significantly enhanced by advances in noninvasive monitoring of blood gases, as well as monitoring of brain and organ oxygenation, perfusion, and hemodynamics. Noninvasive methods to assess lung volume and perfusion shows promise but have been limited to complex techniques that have been primarily used in research studies or invasive clinical procedures.

### SUMMARY OF THE INVENTION

As embodied and broadly described herein, an aspect of the present disclosure relates to a sensor integrated block (SIB) system for real-time respiratory data comprising: a chamber comprising an inside, an inlet and outlet for air, wherein two or more first sensors are in fluid communication with the inside of the chamber, wherein the two or more sensors are selected from an oxygen sensor (2), a carbon dioxide sensor (3), a pressure sensor (4), and a temperature sensor (5); one or more second sensors selected from an SpO₂ sensor (6) or a PaCO₂ sensor; and a processor connected to each of the first and second sensors, and wherein the SIB system measures in real-time respiratory pressure and the inhalation and exhalation volumes of each breath of a patient. In one aspect, the partial pressure of O₂ and CO₂ during the breath, the integrated quantity of oxygen added to the blood, and the integrated quantity of CO₂ removed from the blood during each breath are determined. In another aspect, the SIB system analyzes, displays, and reports patient data to enable a provider to expedite diagnostic and therapeutic decisions and medical evaluations in real-time. In another aspect, the SIB system enables detection of physiologic and physical obstructions that might be preventing optimal oxygen-carbon dioxide exchange at the alveolar level, which can expedite diagnostic and therapeutic pulmonary hygienic intervention. In another aspect, the SIB system can be connected between an air pump or respirator and a mask. In another aspect, the SIB system chamber is integral with a mask. In another aspect, the SIB system chamber is integral with an air pump or respirator. In another aspect, the system further comprises a display connected to the processor, wherein the display shows patient data in an aggregated or disaggregated graphic. In another aspect, the processor and the first, the second, or both the first and second sensors are wired or wireless. In another aspect, the input and output of the chamber each connect to an input and an output hose, respectively. In another aspect, the first sensors comprise 3 or 4 of the sensors.

As embodied and broadly described herein, an aspect of the present disclosure relates to a method of obtaining real-time respiratory data comprising: providing a device capable of connecting to a subject for obtaining the real-time respiratory data, the device comprising: a chamber comprising an inside, an inlet and outlet for air, wherein two or more first sensors are in fluid communication with the inside of the chamber, wherein the two or more sensors are selected from an oxygen sensor (2), a carbon dioxide sensor (3), a pressure sensor (4), and a temperature sensor (5); and one or more second sensors selected from an SpO₂ sensor (6) or a PaCO₂ sensor; and a processor connected to each of the first and second sensors, and wherein the SIB system measures in real-time respiratory pressure and the inhalation and exhalation volumes of each breath of a patient; and calculating the real-time respiratory data with the processor. In one aspect, the partial pressure of O₂ and CO₂ during the breath, the integrated quantity of oxygen added to the blood, and the integrated quantity of CO₂ removed from the blood during each breath are determined. In another aspect, the SIB system analyzes, displays, and reports patient data to enable a provider to expedite diagnostic and therapeutic decisions and medical evaluations in real-time. In another aspect, the SIB system enables detection of physiologic and physical obstructions that might be preventing optimal oxygen-carbon dioxide exchange at the alveolar level, which can expedite diagnostic and therapeutic pulmonary hygienic intervention. In another aspect, the SIB system can be connected between an air pump or respirator and a mask. In another aspect, the SIB system chamber is integral with a mask. In another aspect, the SIB system chamber is integral with an air pump or respirator. In another aspect, the method further comprises providing a display connected to the processor, wherein the display shows patient data in an aggregated or disaggregated graphic. In another aspect, the processor and the first, the second, or both the first and second sensors are wired or wireless. In another aspect, the input and output of the chamber each connect to an input and an output hose, respectively. In another aspect, the first sensors comprise 3 or 4 of the sensors.

As embodied and broadly described herein, an aspect of the present disclosure relates to a method of determining the effectiveness of a pulmonary therapy, the method comprising: (a) measuring real-time respiratory data comprising: providing a device capable of connecting to a subject for obtaining the real-time respiratory data, the device comprising: a chamber comprising an inside, an inlet and outlet for air, wherein two or more first sensors are in fluid communication with the inside of the chamber, wherein the two or more sensors are selected from an oxygen sensor (2), a carbon dioxide sensor (3), a pressure sensor (4), and a temperature sensor (5); one or more second sensors selected from an SpO₂ sensor (6) or a PaCO₂ sensor; and a processor connected to each of the first and second sensors, and wherein the SIB system measures in real-time respiratory pressure and the inhalation and exhalation volumes of each breath of a patient; and calculating the real-time respiratory data with the processor; (b) administering a candidate drug to a first subset of the patients, and a placebo to a second subset of the patients; (c) generating the real-time respiratory data from the first and second subset of patients; (d) calculating a difference between the real-time respiratory data in the first and second subset of patients; and (e) if real-time respiratory data differs between the first and second subset of patients then calculating an effectiveness of the pulmonary therapy.

As embodied and broadly described herein, an aspect of the present disclosure relates to a method of measuring respiratory function, the method comprising: connecting a device capable of connecting to a subject for obtaining the real-time respiratory data, the device comprising: a mask or chamber comprising an inside, an inlet and outlet for air, wherein two or more first sensors are in fluid communication with the inside of the chamber, wherein the two or more sensors are selected from an oxygen sensor (2), a carbon dioxide sensor (3), a pressure sensor (4), and a temperature sensor (5); one or more second sensors selected from an SpO₂ sensor (6) or a PaCO₂ sensor; and a processor connected to each of the first and second sensors, and wherein the SIB system measures in real-time respiratory pressure and the inhalation and exhalation volumes of each breath of a patient; and calculating the real-time respiratory data with the processor to determine respiratory function.

### BRIEF DESCRIPTION OF THE DRAWINGS

For a more complete understanding of the features and advantages of the present invention, reference is now made to the detailed description of the invention along with the accompanying figures and in which:

FIG. 1 is a block diagram of the physical system including one possible layout of sensors mounted to a ‘body’ that connects to standard medical air tubing.

FIG. 2 is a block diagram of the electrical components and their interconnections, including external systems (e.g., a processor, a handheld device, or a computer) to extract and manipulate data, display graphs and save data.

FIG. 3 shows one example of the present invention in which the SIB system is attached between a standard positive airway pressure system.

FIG. 4 shows another example of the present invention in which the SIB system is attached between a standard positive airway pressure system.

FIG. 5 is a schematic diagram of one example of electronics for use with the present invention.

FIG. 6 shows one example of a graphical user interface for use with the present invention.

FIG. 7 shows another example of a graphical user interface for use with the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention is a Sensor Integration Block (SIB) that can be inserted in-line between a patent's respiratory mask and any commercially available respirator or breathing device. This SIB records, logs, and analyzes a comprehensive dataset, and display this data to medical personnel in a manner that assists them with their diagnostic and therapeutic decisions and their medical evaluations in real time. The SIB can also be used as an educational module in a simulation scenario. The SIB can be used independently, that is, solely as a monitoring device. Thus, the SIB can be used as a sensor array in which a mask, that a patient wears for a brief or extended period of time, collects data pertaining to respiratory functioning, which monitors respiratory function. The monitoring device can be used to measure the patient's respiratory functioning when breathing room air naturally.

The system of the present invention displays and records, during each respiratory cycle of inhalation and exhalation, not only the tidal volume and the respiratory pressure, but also the total oxygen and carbon dioxide exchange with the blood in each breath. The system provides a direct measurement of, and the change over time in, the immediate change in blood oxygenation well before the paO₂ indication responds. In the case of apparently healthy, deeply hypoxic patients, i.e., “silent hypoxia”, this system provides a blood oxygen exchange diagnostic that is not directly associated with tidal volume measurements, providing immediate feedback to the caregiver regarding the patient's significant pulmonary health indicators. This data can also be archived and is retrievable in an easy manner to answer voice-recognized queries rapidly and intuitively.

Thus, the present invention enables (1) obstruction detection by providing real-time feedback of respiratory pressure, inhalation volumes, and exhalation volumes for each breath, (2) real-time monitoring during procedures to improve dosing modification, and (3) the system has advanced display and reporting features that provide comprehensive and comparable patient data.

Another feature of the present invention is that it can be integrated into existing assistive respiratory devices to enable improved sensing capabilities not presently offered in most devices. The ability of the invention to enable detection of physiologic and physical obstructions that might be preventing optimal oxygen-carbon dioxide exchange at the alveolar level and report these events in real-time could, as well as the ability to detect “silent hypoxia” in patients with normal tidal volume that are a critical differentiator for this invention.

As used herein, the phrase “Sensor Integration Block (SIB)” refers to a system that measures, in real-time, the respiratory pressure, the inhalation and exhalation volumes on each breath, the variation in the partial pressure of O₂ and CO₂ during the breath, and the integrated quantity of oxygen added to the blood, and the integrated quantity of CO₂ removed from the blood, during each breath. This information may be used to infer physiologic and/or physical obstructions that prevent optimal oxygen-carbon dioxide exchange at the alveolar level of gas exchange, thus enhancing and accelerating diagnostic and therapeutic intervention for pulmonary hygiene such as suctioning, incentive spirometry, percussion, vibration, postural drainage, and/or breathing exercises.

The SIB provides various reporting functions to assure that the current health, and immediate health history, are available to caregivers on various time scales. These reports are described in more detail hereinbelow.

In real-time, during patient procedures and/or the dosing of patient medication, the caregiver will be able to see the immediate level, and change of level over the last few breaths, of the level of gas exchange to the blood during each respiratory cycle. This will also include tidal volume variations and respiratory pressure, and an indicator of how these quantities are trending. This information is useful for making changes in the patient's care and medications in real time, before slower indicators, such as paO₂, respond.

Over the course of minutes, the general trend in the patient's respiratory efficiency can be displayed in an intuitive, easily understood plot. The caregiver can set alert levels and audible trend alarms to draw attention to sudden changes in these patient trends. This short-term data can be easily compared to the respiratory history of the patient over a much wider time interval using an intuitive interface.

On the change of personnel shifts, the SIB system can provide a comprehensive summary of the patient's respiratory history, along with other vital data and caregiver notes, to assure proper continuity of care and emphasis on significant ‘deltas’ in parameters. These reports can be tailored to the severity of the patient's condition, ranging from sleep apnea monitoring to intubated respirator-supported patients to high altitude pulmonary effects in the field.

In other aspect, the system can continuously assimilate this SIB system data, and provide a regressive analysis of the patient's performance during earlier time intervals. An intuitive interface provides the caregiver with detailed histories and correlations with other vital signs over a period that is specified by the caregiver. The system can compare to other cohort patient populations, and provide an easily understood comparison to the patient's respiratory performance to others within the patient's cohort across the general population. Data is provided in a fully disaggregated manner to an artificially intelligent (AI) system that is capable of detecting more complex health indicators through advanced pattern recognition in large, multi-patient data sets for future display and analysis of the patient's condition by the health care professional.

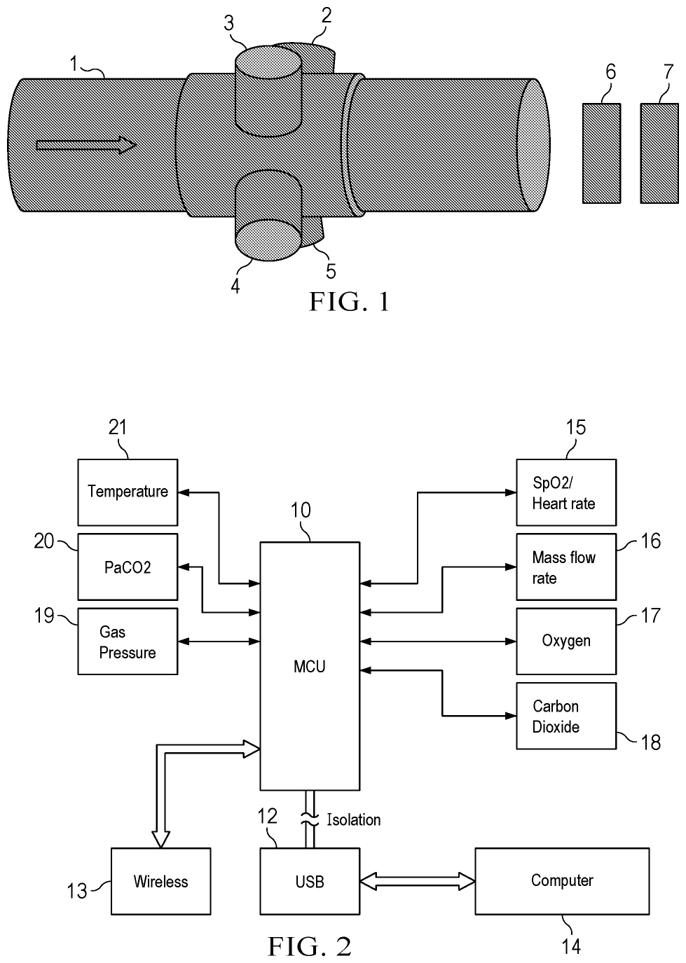

FIG. 1 is a block diagram of the physical system including one possible layout of sensors mounted to a ‘body’ that connects to standard medical air tubing. Air from the individual being monitored enters from external tubing that connects to a bi-directional mass flow sensor (1). Air then enters the sensor system's main body where it expands to connect with an oxygen sensor (2), a carbon dioxide sensor (3), a pressure sensor (4), and a temperature sensor (5). Simultaneously, an SpO₂ sensor (6) and a PaCO₂ sensor (7) are monitored that are either attached to the individual's ear or finger to monitor heart rate, oxygen saturation and PaCO₂ connected to the same host device (e.g., a processor, a handheld device, or a computer), or to different devices that are interconnected, which communications can be wired or wireless.

FIG. 2 is a block diagram of the electrical components and their interconnections, including external systems (e.g., a processor, a handheld device, or a computer) to extract and manipulate data, display graphs and save data. A generic microcontroller (10) communicates with the external processing and display system (14) via any standard wired communication protocol such as USB (12) or a RS-485, RS-232, etc., or via any standard wireless data protocol such as Bluetooth, Wi-Fi, Zigbee, etc. The microcontroller (10) collects sensor data in analog form, or digitized form, from: an SpO₂/heart rate sensor (15), a mass flow sensor (16), an oxygen sensor (17), a carbon dioxide sensor (18), a pressure sensor (19), a PaCO₂ sensor (20), and a temperature sensor (21). Data sampling time can be periodic or arbitrary within the bounds of the microcontroller(s) (10) and the attached sensor devices (15, 16, 17, 18, 19, 20, 21) frequency limitations, which allows flexibility in choosing the desired temporal resolution for monitoring the various parameters during each breathing cycle.

An external processing and display system contains advanced algorithms that computes derived parameters from the fundamental quantities measured by the sensors. A Graphical User Interface (GUI) displays desired data in the form of graphs as well as the real-time collected data. Data is saved on the processing and display unit in a format chosen by the user such as the Comma Separated Value (CSV) file format.

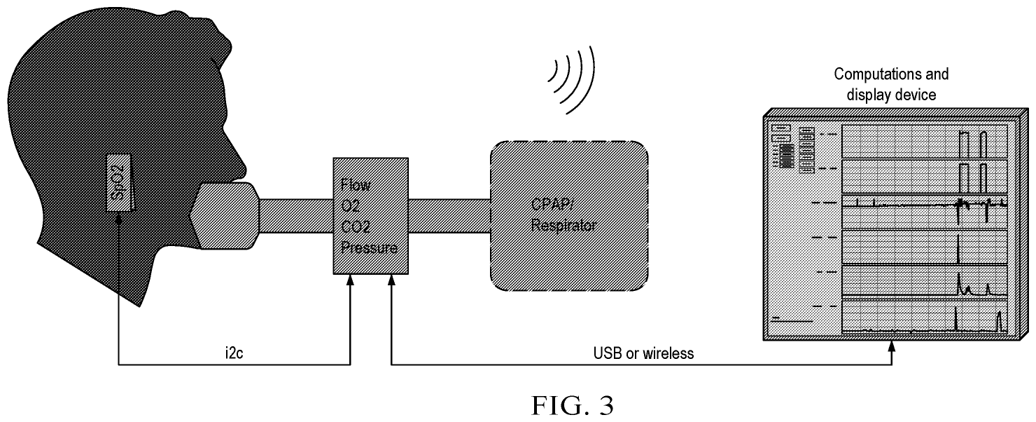

FIG. 3 shows one version of the present invention in which the SIB system is attached between standard positive airway pressure systems such as a continuous positive airway pressure (CPAP), an automatic positive airway pressure (APAP), a Bilevel, or variable positive airway pressure (VPAP), a respirator, or equivalent, and the patient mask. In some examples, the SIB system of the present invention can be integrated into the mask, the tubing, or even at the air pump device. In this example, the SpsO₂ sensor is connected to, e.g., an ear of the patient. These connections can be wired and/or wireless.

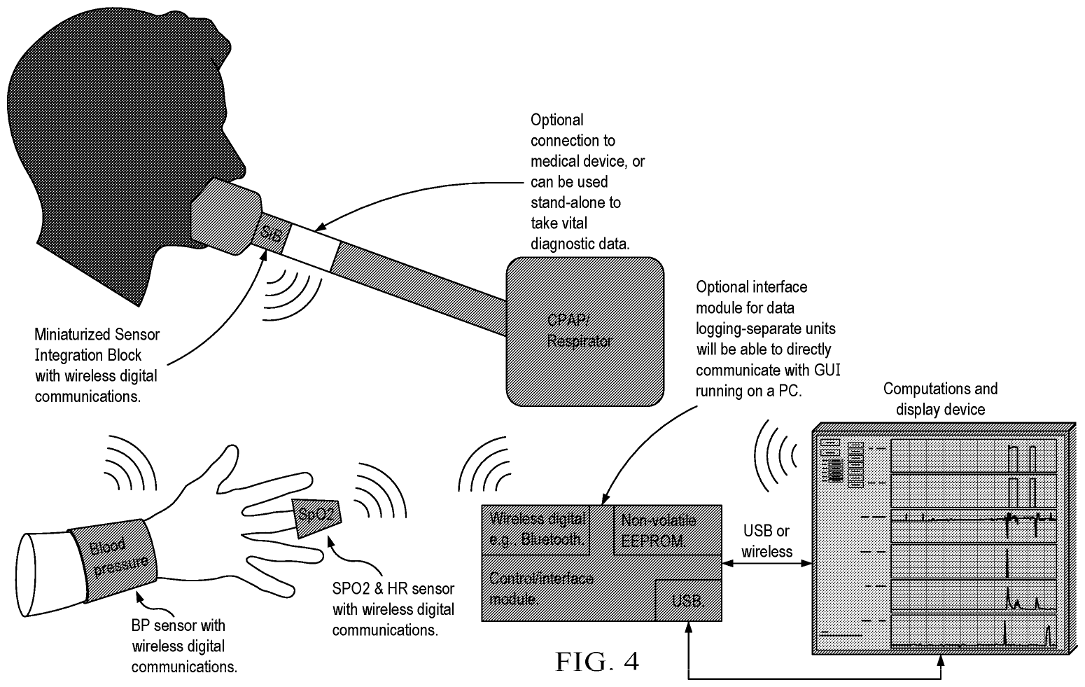

Alternatively, as shown in FIG. 4, it is possible to use separate “autonomous” units for BP, SpO₂ and the gas measurements allow for easily acquiring data from sensors with highly disparate sample rates as well. The incorporation of all these sensors means that nearly every important parameter of a person's present state of health is collected (and recorded) by diagnostic equipment, with the only addition to the “standard” apparatus currently being the addition of a small mask the patient wears. All the parameters are collected in one place, with accurate timestamps. From the data itself, the attending clinician can see at a glance the most important vitals, and more in-depth algorithms can calculate derived parameters. The SIB can be used independently, that is, solely as a monitoring device. Thus, the SIB can be used as a sensor array in which a mask, that a patient wears for a brief or extended period of time, collects data pertaining to respiratory functioning, which monitors respiratory function. The monitoring device can be used to measure the patient's respiratory functioning when breathing room air naturally.

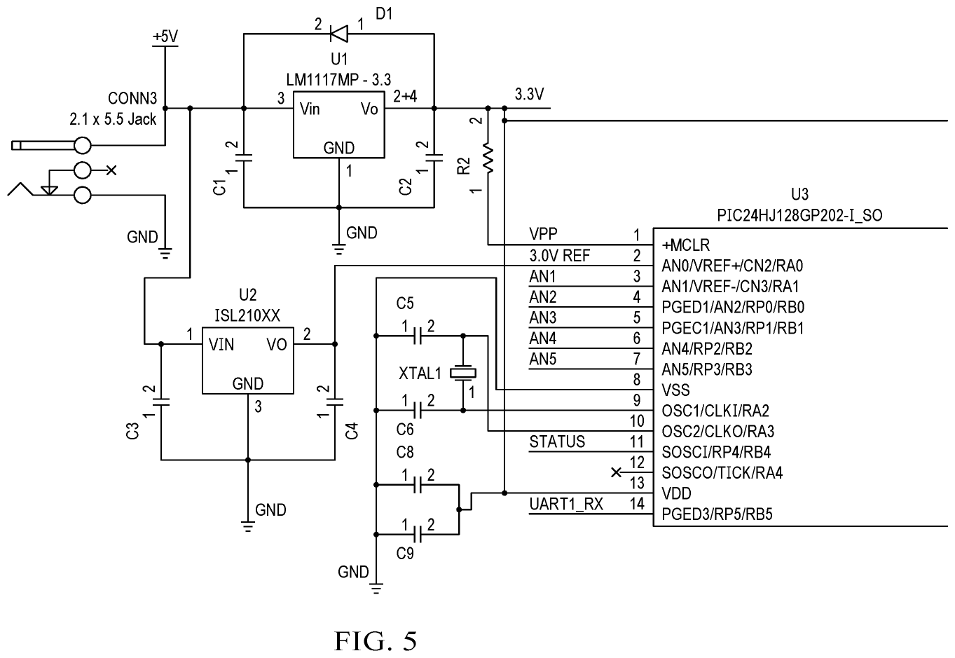

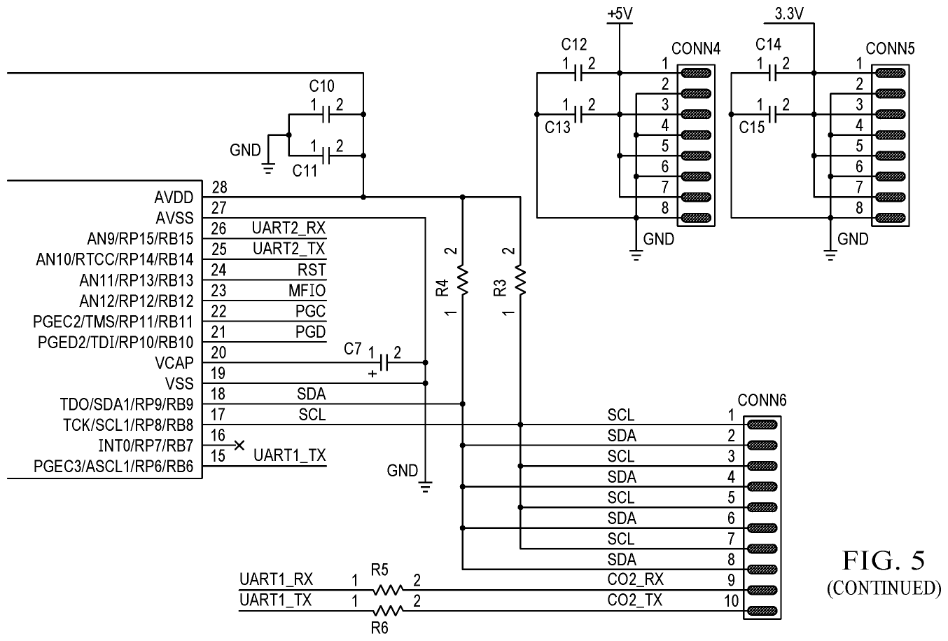

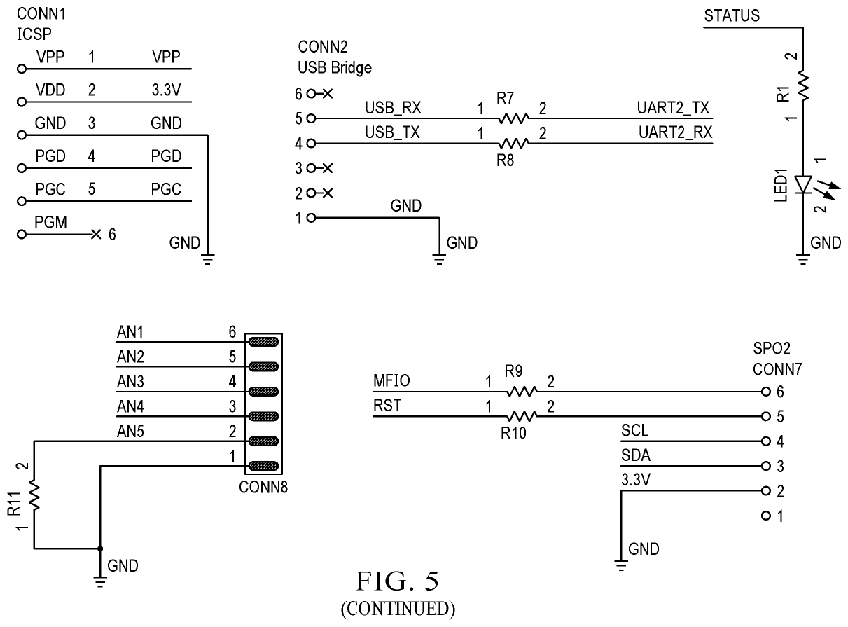

FIG. 5 is a schematic diagram of one example of electronics for use with the present invention. This schematic matches the inputs shown in FIG. 2.

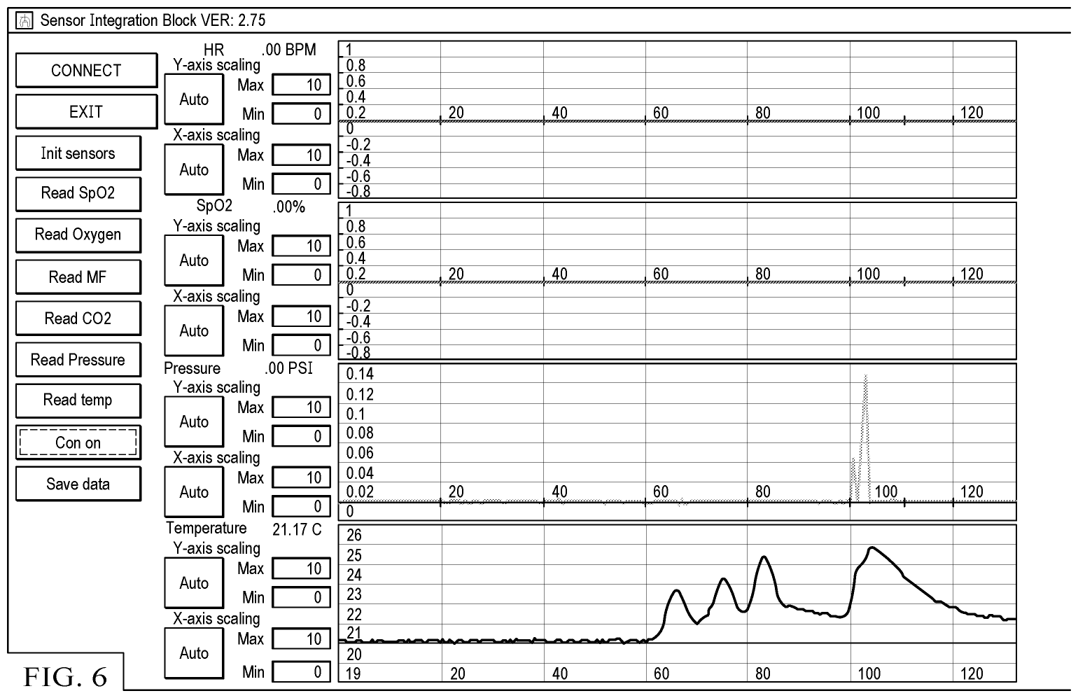

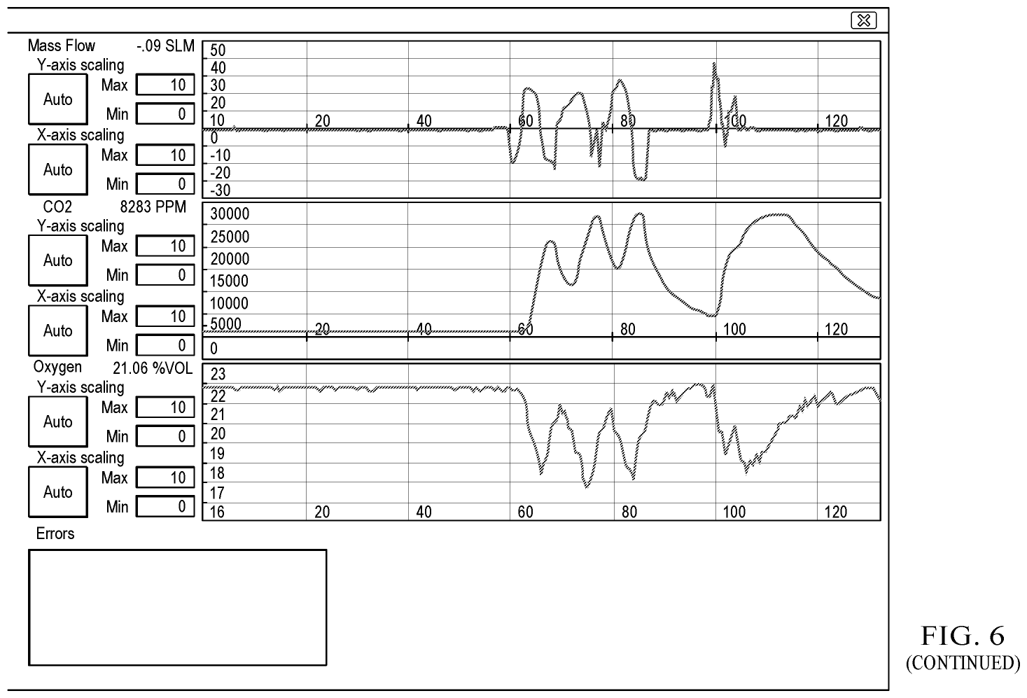

FIG. 6 shows one example of a graphical user interface for use with the present invention. This example shows the various data (an SpO₂/heart rate sensor (15), a mass flow sensor (16), an oxygen sensor (17), a carbon dioxide sensor (18), a pressure sensor (19), a PaCO₂ sensor (20), and a temperature sensor (21)) in separate graphs that allows for the clinician to monitor each individual data stream separately.

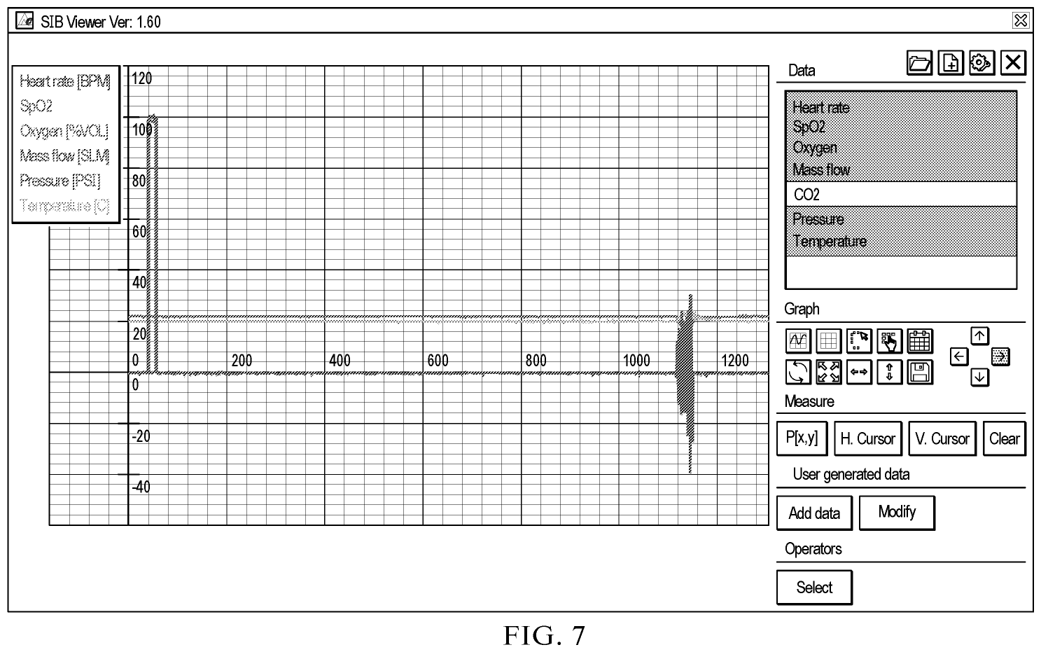

FIG. 7 shows another example of a graphical user interface for use with the present invention. In this example, the various data (an SpO₂/heart rate sensor (15), a mass flow sensor (16), an oxygen sensor (17), a carbon dioxide sensor (18), a pressure sensor (19), a PaCO₂ sensor (20), and a temperature sensor (21)) on the same graph that allows for the clinician to monitor the aggregated data streams.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A sensor integrated block (SIB) system for real-time respiratory data comprising:
a chamber comprising an inside, an inlet and outlet for air, wherein two or more first sensors are in fluid communication with the inside of the chamber, wherein the two or more sensors are selected from an oxygen sensor (2), a carbon dioxide sensor (3), a pressure sensor (4), and a temperature sensor (5);
one or more second sensors selected from an SpO₂ sensor (6) or a PaCO₂ sensor; and
a processor connected to each of the first and second sensors, and wherein the SIB system measures in real-time respiratory pressure and the inhalation and exhalation volumes of each breath of a patient.

2. The system of claim 1, wherein the partial pressure of O₂ and CO₂ during the breath, the integrated quantity of oxygen added to the blood, and the integrated quantity of CO₂ removed from the blood during each breath are determined.

3. The system of claim 1, wherein the SIB system analyzes, displays, and reports patient data to enable a provider to expedite diagnostic and therapeutic decisions and medical evaluations in real-time.

4. The system of claim 1, wherein the SIB system enables detection of physiologic and physical obstructions that might be preventing optimal oxygen-carbon dioxide exchange at the alveolar level, which can expedite diagnostic and therapeutic pulmonary hygienic intervention.

5. The system of claim 1, wherein at least one of:
the SIB system can be connected between an air pump or respirator and a mask;
the SIB system chamber is integral with a mask; or
the SIB system chamber is integral with an air pump or respirator.

6. (canceled)

7. (canceled)

8. The system of claim 1, further comprising a display connected to the processor, wherein the display shows patient data in an aggregated or disaggregated graphic.

9. The system of claim 1, wherein the processor and the first, the second, or both the first and second sensors are wired or wireless.

10. The system of claim 1, wherein the input and output of the chamber each connect to an input and an output hose, respectively.

11. The system of claim 1, wherein the first sensors comprise 3 or 4 of the sensors.

12. A method of obtaining real-time respiratory data comprising:
providing a device capable of connecting to a subject for obtaining the real-time respiratory data, the device comprising:
a chamber comprising an inside, an inlet and outlet for air, wherein two or more first sensors are in fluid communication with the inside of the chamber, wherein the two or more sensors are selected from an oxygen sensor (2), a carbon dioxide sensor (3), a pressure sensor (4), and a temperature sensor (5); and
one or more second sensors selected from an SpO₂ sensor (6) or a PaCO₂ sensor; and
a processor connected to each of the first and second sensors, and wherein the SIB system measures in real-time respiratory pressure and the inhalation and exhalation volumes of each breath of a patient; and
calculating the real-time respiratory data with the processor.

13. The method of claim 12, wherein the partial pressure of O₂ and CO₂ during the breath, the integrated quantity of oxygen added to the blood, and the integrated quantity of CO₂ removed from the blood during each breath are determined.

14. The method of claim 12, wherein the SIB system analyzes, displays, and reports patient data to enable a provider to expedite diagnostic and therapeutic decisions and medical evaluations in real-time.

15. The method of claim 12, wherein the SIB system enables detection of physiologic and physical obstructions that might be preventing optimal oxygen-carbon dioxide exchange at the alveolar level, which can expedite diagnostic and therapeutic pulmonary hygienic intervention.

16. The method of claim 12, wherein at least one of:
the SIB system can be connected between an air pump or respirator and a mask;
the SIB system chamber is integral with a mask; or
the SIB system chamber is integral with an air pump or respirator.

17. (canceled)

18. (canceled)

19. The method of claim 12, further comprising providing a display connected to the processor, wherein the display shows patient data in an aggregated or disaggregated graphic.

20. The method of claim 12, wherein the processor and the first, the second, or both the first and second sensors are wired or wireless.

21. The method of claim 12, wherein the input and output of the chamber each connect to an input and an output hose, respectively.

22. The method of claim 12, wherein the first sensors comprise 3 or 4 of the sensors.

23. A method of determining the effectiveness of a pulmonary therapy, the method comprising:
(a) measuring real-time respiratory data comprising:
providing a device capable of connecting to a subject for obtaining the real-time respiratory data, the device comprising:
a chamber comprising an inside, an inlet and outlet for air, wherein two or more first sensors are in fluid communication with the inside of the chamber, wherein the two or more sensors are selected from an oxygen sensor (2), a carbon dioxide sensor (3), a pressure sensor (4), and a temperature sensor (5);
one or more second sensors selected from an SpO₂ sensor (6) or a PaCO₂ sensor; and
a processor connected to each of the first and second sensors, and wherein the SIB system measures in real-time respiratory pressure and the inhalation and exhalation volumes of each breath of a patient; and
calculating the real-time respiratory data with the processor;
(b) administering a candidate drug to a first subset of the patients, and a placebo to a second subset of the patients;
(c) generating the real-time respiratory data from the first and second subset of patients;
(d) calculating a difference between the real-time respiratory data in the first and second subset of patients; and
(e) if real-time respiratory data differs between the first and second subset of patients then calculating an effectiveness of the pulmonary therapy.

24. A method of measuring respiratory function, the method comprising:
connecting a device capable of connecting to a subject for obtaining the real-time respiratory data, the device comprising:
a mask or chamber comprising an inside, an inlet and outlet for air, wherein two or more first sensors are in fluid communication with the inside of the chamber, wherein the two or more sensors are selected from an oxygen sensor (2), a carbon dioxide sensor (3), a pressure sensor (4), and a temperature sensor (5);
one or more second sensors selected from an SpO₂ sensor (6) or a PaCO₂ sensor; and
a processor connected to each of the first and second sensors, and wherein the SIB system measures in real-time respiratory pressure and the inhalation and exhalation volumes of each breath of a patient; and
calculating the real-time respiratory data with the processor to determine respiratory function.
